(19) United States
(12) Patent Application Publication
(10) Pub. No.: US 2025/0248614 A1
(43) Pub. Date: Aug. 7, 2025

(54) CEREBRAL BLOOD FLOW (CBF) CORRECTION METHOD BASED ON MULTIPLE POST-LABELING DELAYS (PLDS), SYSTEM, AND MEDIUM

(71) Applicant: Anying Technology (Beijing) Co., Ltd., Beijing (CN)

(72) Inventors: Gang MENG, Beijing (CN); Ligeng LI, Beijing (CN); Mingyang ZHAO, Beijing (CN); Xiangyu GONG, Beijing (CN); Lihua ZHONG, Beijing (CN)

(73) Assignee: Anying Technology (Beijing) Co., Ltd., Beijing (CN)

(21) Appl. No.: 18/758,829

(22) Filed: Jun. 28, 2024

(30) Foreign Application Priority Data
Feb. 4, 2024 (CN) 202410160123.5

(51) Int. Cl. A61B 5/026; A61B 5/00; A61B 5/055; G01R 33/56; G01R 33/563; G06T 7/00; G06T 7/11

(52) U.S. Cl. CPC A61B 5/0263; A61B 5/0042; A61B 5/055; G01R 33/5608; G01R 33/56366; G06T 7/0012; G06T 7/11; G06T 2207/10088; G06T 2207/30016; G06T 2207/30104

(57) ABSTRACT

The invention relates to a cerebral blood flow (CBF) correction method based on multiple post-labeling delays (PLDs), a system, and a non-transitory computer-readable storage medium, which relate to CBF detection. The CBF correction method based on multiple PLDs includes importing a CBF perfusion image and an arterial transit time (ATT) image corresponding to the CBF perfusion image into a structure space to obtain a CBF perfusion model, the CBF perfusion image including multiple PLDs; registering a brain atlas to the CBF perfusion model to obtain a brain segmented CBF perfusion model; and taking, in the brain segmented CBF perfusion model, a highest CBF in multiple PLDs corresponding to each region as a corrected CBF of the each region. According to the CBF correction method, the CBF is obtained more accurately.

### CROSS REFERENCE TO RELATED APPLICATION

This patent application claims the benefit and priority of Chinese Patent Application No. 2024101601235, filed with the China National Intellectual Property Administration on Feb. 4, 2024, the disclosure of which is incorporated by reference herein in its entirety as part of the present application.

### BACKGROUND OF THE INVENTION

### Field of the Invention

At least one embodiment relates to the technical field of cerebral blood flow (CBF) detection, and in particular to a CBF correction method based on a multiple post-labeling delays (PLDs), a system, and a medium.

### Description of the Related Art

In CBF calculation, according to existing concepts, a PLD corresponding to arterial transit time (ATT) is searched mostly, so as to search for a more real CBF corresponding to the ATT. In an article, a method for obtaining the CBF corresponding to the PLD slightly later than the ATT is presented. While accurately calculating the CBF of a specific region of the brain tissue, this method underestimates the CBF in other regions. Hence, the accuracy for obtaining the CBF is to be improved.

### BRIEF SUMMARY OF THE INVENTION

An objective of at least one embodiment of the invention is to provide a CBF correction method based on multiple PLDs, a system, and a medium, to obtain a CBF more accurately.

To achieve the above objective, at least one embodiment provides the following technical solutions.

A CBF correction method based on multiple PLDs includes:
importing a CBF perfusion image and an ATT image corresponding to the CBF perfusion image into a structure space to obtain a CBF perfusion model, the CBF perfusion image including multiple PLDs;
registering a brain atlas to the CBF perfusion model to obtain a brain segmented CBF perfusion model; and
taking, in the brain segmented CBF perfusion model, a highest CBF in multiple PLDs corresponding to each region as a corrected CBF of each region.

Optionally, the CBF correction method based on multiple PLDs further includes: taking ATT corresponding to the highest CBF in the multiple PLDs corresponding to each region as optimal ATT of each region.

Optionally, the CBF correction method based on multiple PLDs further includes: determining, if optimal ATT of a region i is greater than 1.3 times of preset ATT of the region i, that the region i has collateral circulation.

Optionally, before the importing a CBF perfusion image and an ATT image corresponding to the CBF perfusion image into a structure space to obtain a CBF perfusion model, the CBF correction method based on multiple PLDs further includes:
acquiring the CBF perfusion image and the ATT image from an individual brain by multi-delay pseudo-continuous arterial spin labeling (pCASL).

Optionally, the structure space is a T2 Flair space.

Optionally, the brain atlas includes an AAL3 atlas and a lobe atlas in an MNI152 space.

Optionally, the multiple PLDs include five PLDs, and the five PLDs occur at 0.5 s, 1.0 s, 1.5 s, 2 s and 2.5 s sequentially, or the multiple PLDs include any two or three of 1.0 s, 1.5 s, 2 s and 2.5 s.

At least one embodiment further provides a computer system, including a memory, a processor and a computer program stored in the memory and executable on the processor, where the processor executes the computer program to implement the steps of the CBF correction method based on multiple PLDs.

At least one embodiment further provides a computer-readable storage medium, where the computer-readable storage medium stores a computer program, and the computer program is executed by a processor to implement the steps of the CBF correction method based on multiple PLDs.

According to one or more embodiments of the invention, at least one embodiment has the following technical effects:

By taking the highest CBF in the multiple corresponding PLDs of each brain segmented region as the corrected CBF of the region, the at least one embodiment of the invention solves the problem that the existing arterial spin labeling (ASL) cerebral perfusion imaging is limited to a single timepoint and a single parameter to underestimate the CBF, and obtains the CBF more accurately.

### BRIEF DESCRIPTION OF THE DRAWINGS

To describe the technical solutions in one or more embodiments of the invention or in the prior art more clearly, the accompanying drawings required for the embodiments are briefly described below. Apparently, the accompanying drawings in the following description show merely some embodiments of the invention, and those of ordinary skill in the art may still derive other accompanying drawings from these accompanying drawings without creative efforts.

FIG. 1 illustrates a flowchart of a CBF correction method based on multiple PLDs according to one or more embodiments of the invention; and

FIG. 2 illustrates the internal structure of a computer system, according to one or more embodiments of the invention.

### DETAILED DESCRIPTION OF THE INVENTION

The technical solutions of the one or more embodiments of the invention are clearly and completely described below with reference to the drawings in the one or more embodiments of the invention. Apparently, the described one or more embodiments are merely a part rather than all of the embodiments of the invention. All other embodiments obtained by those skilled in the art based on the one or more embodiments of the invention without creative efforts shall fall within the protection scope of the invention.

An objective of at least one embodiment of the invention is to provide a CBF correction method based on multiple PLDs, a system, and a medium, to obtain a CBF more accurately.

In order to make the above objective, features and advantages of the one or more embodiments of the invention clearer and more comprehensible, the one or more embodiments will be further described in detail below in combination with accompanying drawings and particular implementation modes.

### Example 1

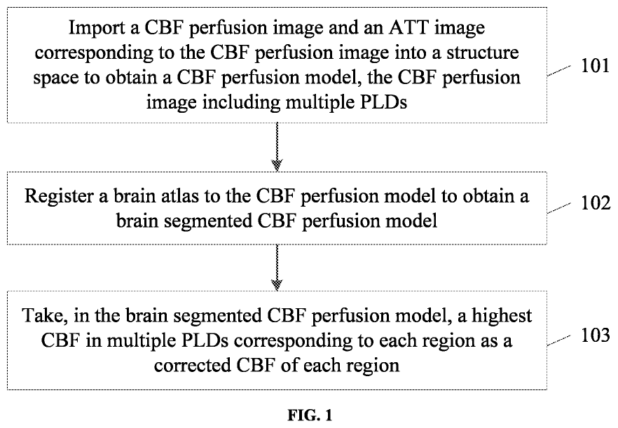

As shown in FIG. 1, according to one or more embodiments, a CBF correction method based on multiple PLDs includes the following steps:
Step 101: A CBF perfusion image and an ATT image corresponding to the CBF perfusion image are imported into a structure space to obtain a CBF perfusion model, the CBF perfusion image including multiple PLDs.
Step 102: A brain atlas is registered to the CBF perfusion model to obtain a brain segmented CBF perfusion model.
Step 103: In the brain segmented CBF perfusion model, the highest CBF in multiple PLDs corresponding to each region is a corrected CBF of each region.

The CBF correction method based on multiple PLDs further includes: ATT corresponding to the highest CBF in the multiple PLDs corresponding to each region is taken as optimal ATT of each region.

If optimal ATT of a region i is greater than 1.3 times of preset ATT of the region i, the region i has collateral circulation. Upon the determination of the collateral circulation in the region, there exists compensatory circulation. The preset ATT of the region i is the standard ATT of the region i.

The CBF is milliliters of blood per 100 g of brain tissue per minute.

Before step 101, the CBF correction method based on multiple PLDs further includes: The CBF perfusion image is acquired from an individual brain by multi-delay pCASL, and the ATT image corresponding to the CBF perfusion image is acquired at the same time.

In at least one embodiment, with a new-generation 4DASL (multi-delay pCASL) scanning sequence and a Cereflow efficient computing platform system, limitations of the ASL technique are solved, and CBF correction computation on 300 target regions of the whole brain for 30-90 s is realized.

The multiple PLDs include five PLDs. The five PLDs occur at 0.5 s, 1.0 s, 1.5 s, 2 s and 2.5 s sequentially. Or the multiple PLDs include any two or three of 1.0 s, 1.5 s, 2 s and 2.5 s. That is, there may be five PLDs, and may also be two or three PLDs.

The structure space is a T2 Flair space. The CBF perfusion model is a three-dimensional (3D) model.

The brain atlas includes an AAL3 atlas and a lobe atlas in an MNI152 space. The brain atlas is used to segment the brain tissue. Each segmented region includes a parietal lobe, a frontal lobe, an occipital lobe, a temporal lobe, etc. For each region, a size of a minimum segmented unit can be determined according to an actual need. Same as the corrected CBF of each region, a corrected CBF and optimal ATT of the minimum segmented unit are determined.

In the step 103, the corrected CBF of each segmented region in the brain tissue is output at last.

The ATT falls in a range of 0.7-1.3 s, so the ATT of each brain region is different significantly. Determining the CBF of the whole brain based on some PLD causes the CBF underestimating problem. According to one or more embodiments, the determination of the CBF is not limited to the single timepoint. The ATT corresponding to the highest CBF in the multiple PLDs corresponding to each region is taken as the optimal ATT of each region. That is, the PLD with the highest CBF perfusion level is selected for each brain segmented region. The optimal ATT and the CBF of the brain segmented region are determined according to the PLD with the highest CBF perfusion level. The at least one embodiment of the invention obtains the CBF more accurately.

### Example 2

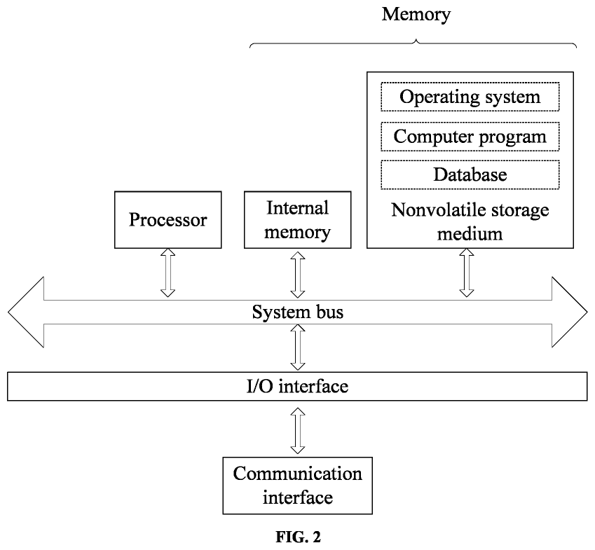

At least one embodiment provides a computer system. The internal structure of the computer system may be as shown in FIG. 2, according to one or more embodiments of the invention. The computer system includes a processor, a memory, an input/output (I/O) interface, and a communication interface. The processor, the memory and the I/O interface are connected through a system bus. The communication interface is connected to the system bus through the I/O interface. The processor of the computer system is configured to provide computing and control capabilities. The memory of the computer system includes a nonvolatile storage medium and an internal memory. The nonvolatile storage medium stores an operating system, a computer program and a database. The internal memory provides an environment for the operation of the operating system and the computer program in the nonvolatile storage medium. The database of the computer system is configured to store an event to be processed. The I/O interface of the computer system is configured to exchange information between the processor and an external device. The communication interface of the computer system is configured to communicate with an external terminal through a network. The computer program is executed by the processor to implement the CBF correction method based on multiple PLDs by way of one or more embodiments of the invention.

### Example 3

At least one embodiment of the invention further provides a non-transitory computer-readable storage medium. The non-transitory computer-readable storage medium stores a computer program. The computer program is executed by a processor to implement the steps of the CBF correction method based on multiple PLDs by way of one or more embodiments of the invention.

The technical characteristics of the one or more embodiments can be employed in arbitrary combinations. To provide a concise description of these embodiments, all possible combinations of all the technical characteristics of the one or more embodiments may not be described; however, these combinations of the technical characteristics should be construed as falling within the scope defined by the specification as long as no contradiction occurs.

Particular examples are used herein for illustration of principles and implementation modes of the one or more embodiments of the invention. The descriptions of the one or more embodiments are merely used for assisting in understanding the method of the invention and its core ideas. In addition, those of ordinary skill in the art can make various modifications in terms of particular implementation modes and the scope of application in accordance with the ideas of the one or more embodiments of the invention. In conclusion, the content of the description shall not be construed as limitations to the invention.

1. A cerebral blood flow (CBF) correction method based on multiple post-labeling delays (PLDs), comprising:
importing a CBF perfusion image and an arterial transit time (ATT) image corresponding to the CBF perfusion image into a structure space to obtain a CBF perfusion model, the CBF perfusion image comprising multiple PLDs;
registering a brain atlas to the CBF perfusion model to obtain a brain segmented CBF perfusion model; and
taking, in the brain segmented CBF perfusion model, a highest CBF in said multiple PLDs corresponding to each region as a corrected CBF of said each region.

2. The CBF correction method based on multiple PLDs according to claim 1, further comprising: taking ATT corresponding to the highest CBF in the multiple PLDs corresponding to each region as optimal ATT of each region.

3. The CBF correction method based on multiple PLDs according to claim 2, further comprising: determining, if said optimal ATT of a region i is greater than 1.3 times of preset ATT of the region i, that the region i has collateral circulation.

4. The CBF correction method based on multiple PLDs according to claim 1, before the importing a CBF perfusion image and an ATT image corresponding to the CBF perfusion image into a structure space to obtain a CBF perfusion model, further comprising:
acquiring the CBF perfusion image and the ATT image from an individual brain by multi-delay pseudo-continuous arterial spin labeling (pCASL).

5. The CBF correction method based on multiple PLDs according to claim 1, wherein the structure space is a T2 Flair space.

6. The CBF correction method based on multiple PLDs according to claim 1, wherein the brain atlas comprises an AAL3 atlas and a lobe atlas in an MNI152 space.

7. The CBF correction method based on multiple PLDs according to claim 1, wherein the multiple PLDs comprise five PLDs, the five PLDs occur at 0.5 s, 1.0 s, 1.5 s, 2 s and 2.5 s sequentially, or the multiple PLDs comprise any two or three of 1.0 s, 1.5 s, 2 s and 2.5 s.

8. A computer system, comprising:
a memory,
a processor, and
a computer program stored in the memory and executable on the processor,
wherein the processor executes the computer program to implement a cerebral blood flow (CBF) correction method based on multiple post-labeling delays (PLDs), said CBF correction method comprising
importing a CBF perfusion image and an arterial transit time (ATT) image corresponding to the CBF perfusion image into a structure space to obtain a CBF perfusion model, the CBF perfusion image comprising multiple PLDs;
registering a brain atlas to the CBF perfusion model to obtain a brain segmented CBF perfusion model; and
taking, in the brain segmented CBF perfusion model, a highest CBF in said multiple PLDs corresponding to each region as a corrected CBF of said each region.

9. A non-transitory computer-readable storage medium, wherein the non-transitory computer-readable storage medium stores a computer program, and the computer program is executed by a processor to implement a cerebral blood flow (CBF) correction method based on multiple post-labeling delays (PLDs), said CBF correction method comprising:
importing a CBF perfusion image and an arterial transit time (ATT) image corresponding to the CBF perfusion image into a structure space to obtain a CBF perfusion model, the CBF perfusion image comprising multiple PLDs;
registering a brain atlas to the CBF perfusion model to obtain a brain segmented CBF perfusion model; and
taking, in the brain segmented CBF perfusion model, a highest CBF in said multiple PLDs corresponding to each region as a corrected CBF of said each region.

10. The computer system according to claim 8, further comprising, taking ATT corresponding to the highest CBF in the multiple PLDs corresponding to said each region as an optimal ATT of said each region.

11. The computer system according to claim 10, further comprising, determining, if said optimal ATT of a region i is greater than 1.3 times of preset ATT of the region i, that the region i has collateral circulation.

12. The computer system according to claim 8, before the importing a CBF perfusion image and an ATT image corresponding to the CBF perfusion image into a structure space to obtain a CBF perfusion model, further comprising,
acquiring the CBF perfusion image and the ATT image from an individual brain by multi-delay pseudo-continuous arterial spin labeling (pCASL).

13. The computer system according to claim 8, wherein the structure space is a T2 Flair space.

14. The computer system according to claim 8, wherein the brain atlas comprises an AAL3 atlas and a lobe atlas in an MNI152 space.

15. The computer system according to claim 8, wherein the multiple PLDs comprise five PLDs, the five PLDs occur at 0.5 s, 1.0 s, 1.5 s, 2 s and 2.5 s sequentially, or the multiple PLDs comprise any two or three of 1.0 s, 1.5 s, 2 s and 2.5 s.

16. The non-transitory computer-readable storage medium according to claim 9, further comprising, taking ATT corresponding to the highest CBF in the multiple PLDs corresponding to said each region as an optimal ATT of said each region.

17. The non-transitory computer-readable storage medium according to claim 16, further comprising, determining, if said optimal ATT of a region i is greater than 1.3 times of preset ATT of the region i, that the region i has collateral circulation.

18. The non-transitory computer-readable storage medium according to claim 9, before the importing a CBF perfusion image and an ATT image corresponding to the CBF perfusion image into a structure space to obtain a CBF perfusion model, further comprising,
acquiring the CBF perfusion image and the ATT image from an individual brain by multi-delay pseudo-continuous arterial spin labeling (pCASL).

19. The non-transitory computer-readable storage medium according to claim 9, wherein the structure space is a T2 Flair space.

20. The non-transitory computer-readable storage medium according to claim 9, wherein the brain atlas comprises an AAL3 atlas and a lobe atlas in an MNI152 space.
